(19) United States
(12) Patent Application Publication
(10) Pub. No.: US 2009/0043111 A1
(43) Pub. Date: Feb. 12, 2009

(54) Novel process for ropinirole preparation

(76) Inventor: Meizheng Liu, Columbia, MD (US)

Correspondence Address: Jerome J. Norris, Suite 305, 1901 Pennsylvania Avenue, Washington, DC, 20006, US

(21) Appl. No.: 11/882,806

(22) Filed: Aug. 6, 2007

(51) Int. Cl. C07D 209/34

(52) U.S. Cl. 548/486

(57) ABSTRACT

A method for producing compounds of the formula:

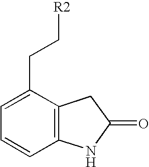

Wherein R2 is selected from the group consisting of amino, lower alkylamino, di-lower alkylamino, allylamino, diallylamino, N-lower alkyl-N-allylamino, benzylamino, dibenzylamino, phenethylamino, diphenethylamino, 4-hydroxyphenethylamino or di-(4-hydroxyphenethylamino);
comprising:
a) reacting

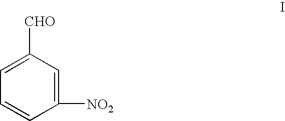

with Wittig Reagent to produce

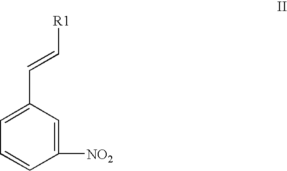

wherein R1 is alkoxyl;
f) hydrolyzing compound II under acidic conditions to produce

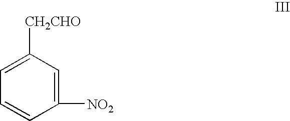

g) subjecting compound III to reduction and amination to produce the tertiary amine

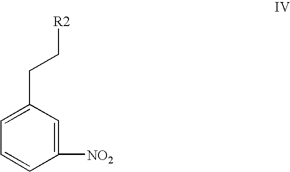

wherein R2 is as defined above;
h) reacting compound IV with
4-chlorophenoxyacetonitrile to create nucleophilic substitution to produce the nitrile compound

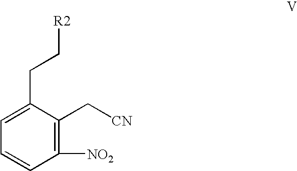

wherein R2 is as defined above; and
i) effecting reduction and hydrolysis in the same step with palladium/C and sodium hydroxide to produce

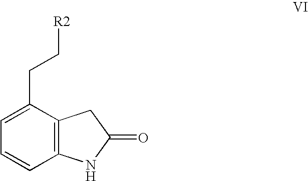

wherein R2 is as defined above.

### FIELD OF THE INVENTION

The present invention relates to a novel process for preparation of ropinirole, 4-[(2-dipropylamino)ethyl]-1,3-dihydro-2H-indol-2-one hydrochloride, a compound used in the treatment of Parkinson's disease, and having the structural formula:

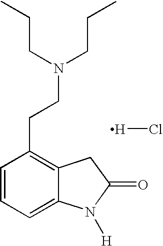

This process is particularly suitable for the production of large quantities of ropinirole as the synthesis is efficient, and therefore more cost effective. Further, the invention process utilizes a shorter route having less reaction steps, from inexpensive and commercially available starting materials than heretofore known processes for producing ropinirole.

### BACKGROUND OF THE INVENTION

### The Prior Art

Processes for the preparation of substituted indolone derivatives, particularly, ropinirole, are known in the prior art.

For example, 4-aminoalky-2-(3H)-indolones are utilized as the D₂-agonist for treatment of hypertension, and are shown in U.S. Pat. No. 4,452,808. These compounds are illustrated by structural formula I as follows:

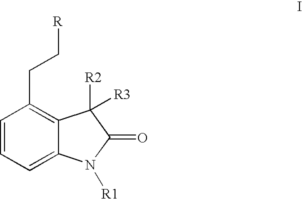

in which:

R is amino, lower alkylamino, di-lower alkylamino, allylamino, diallylamino, N-lower alkyl-N-allylamino, benzylamino, dibenzylamino, phenethylamino, diphenethylamino, 4-hydroxyphenethylamino or di-(4-hydroxyphenethylamino); wherein

R1, R2 or R3 are, each, hydrogen or lower alkyl, (CH₂)ₙ, and n is 1-3.

A subgeneric group comprises the compounds of formula I in which:

R is amino, di-n-propylamino, n-propyl-n-butylamino or 4-hydroxyphenethylamino;

wherein

A preferred species of this invention is 4-(2-di-n-propylaminoethyl)-2(3H)-indolone or one of its pharmaceutically acceptable, acid addition salts.

The term “lower alkyl” used herein is for convenience, to include branched and straight chain groups of from 1-6 carbons, preferably n-propyl, for each alkyl in R and from 1-4 carbons, preferably methyl, for each of R1, R2 and R3. R1, R2 and R3 are preferably, for ease of preparation, all the same.

In the prior art, the reaction sequences for these compounds are as shown in Schemes A and B as follows:

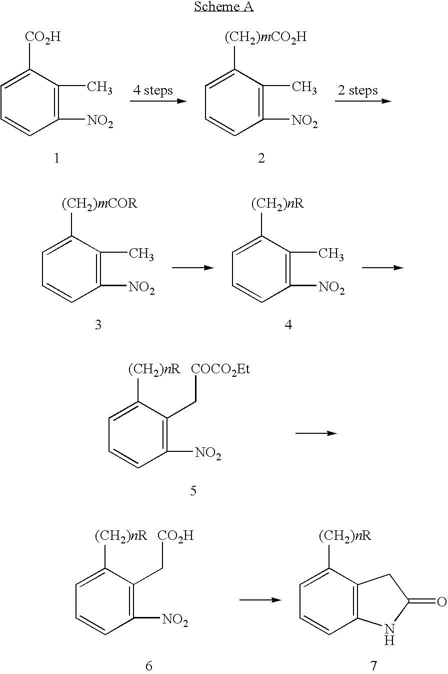

In scheme A, m is n−1 and R is as described in formula I

In some cases, such as where R is a primary or secondary amine function, an amino protective group may be present.

This is amongst the first synthesis of ropinirole, which is also described in J. Med. Chem. 1985, 28, 1533-1538. The synthesis of ropinirole requires a ten-step reaction (going from 1 to 3 in scheme A requires six steps) to produce an over all yield of as low as 23 percent from 2-methyl-3-nitrobenzolic acid. The chemistry of this synthesis is very expensive because of too many steps and the high materials expense. Hence, it is only suitable for preparation of a small quantity of ropinirole.

Another route for preparing ropinirole is disclosed in U.S. Pat. No. 5,336,781 and also published in J. Heterocyclic Chem., 32, 875-882 (1995). It provides in a first aspect, the preparation of a compound of structure (IX).

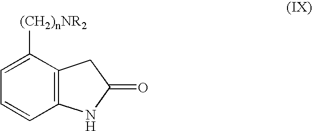

wherein n is 1 to 3 and each group R is a hydrogen or C₁₋₄ alkyl, which comprises, reaction of a compound of the structure (VIII).

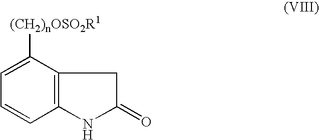

In which n is 1 to 3 and R¹ is C₁₋₄alkyl, phenyl or substituted phenyl with an amine of structure HNR₂ where R is as described for structure (IX), and optionally thereafter forming a salt.

One of the groups R is hydrogen and the other is hydrogen or C₁₋₄ alkyl; preferably, both groups R and C₁₋₄ alkyl, in particular propyl. n is 1 to 3; preferably n is 2.

R₁ is C₁₋₄ alkyl such as methyl, phenyl or substituted phenyl, for example p-methylphenyl; preferably R₁ is p-methylphenyl.

In this second prior art synthesis, the reaction is carried out in a variety of solvents at elevated temperature, preferably in water, at a temperature of between 80° and about 90° C.

In preparing the compounds (and in particular ropinirole) the processes used to prepare the compounds (VIII), proceed via important intermediates. In particular, the compounds (VIII) are prepared via the following route:

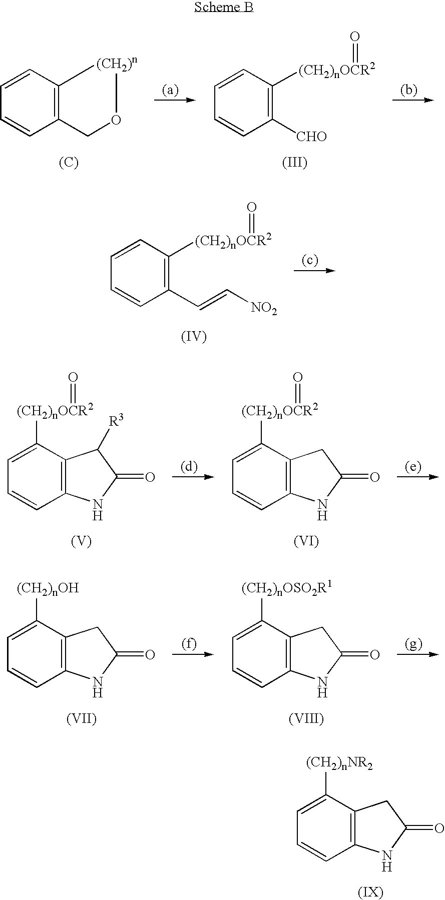

In this route, a seven-step (a-g) reaction sequence arrives at ropinirole in a 24% overall yield from isochroman (C). Also it is suitable for a large scale manufacture of ropinirole because it produces higher overall yield, employing standard processing equipment, and using cheap and readily available reagents. Although this route was shorter than the first one in its essential steps, and more suitable for preparation of kilogram quantities of ropinirole, the starting material, isochroman (C) is very expensive and the final step gives a considerable amount of eliminative by product.

M. Makosza reported, in J. Org. Chem., 1984, 49, 1494-1499, a reaction which is related to direct alpha-cyanoalkylation of nitroarenes by vicarious nucleophilic substitution of hydrogen in nitroarenes with alpha-substituted nitrites and esters. This is shown in the reaction scheme as follows:

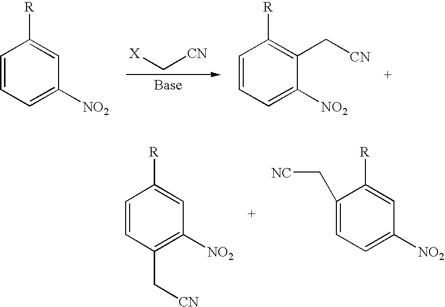

In the above scheme, R is halogen, hydrogen, alkyl, alkoxy, alkylthio, and phenyl group. This reaction usually led to a mixture of isomeric products containing two ortho positions and one para position.
The above sequence appears to have value in forming generic aryl nitrites, but the possibility of using this reaction for ropinirole synthesis is neither known nor contemplated.

The inventive advance of this invention is the use of a controlled cyanomethylation for ropinirole synthesis. Further, in the present invention, only a single isomer was obtained when reaction conditions are specifically chosen.

In general, the compounds of this invention are prepared by the following reaction sequence:

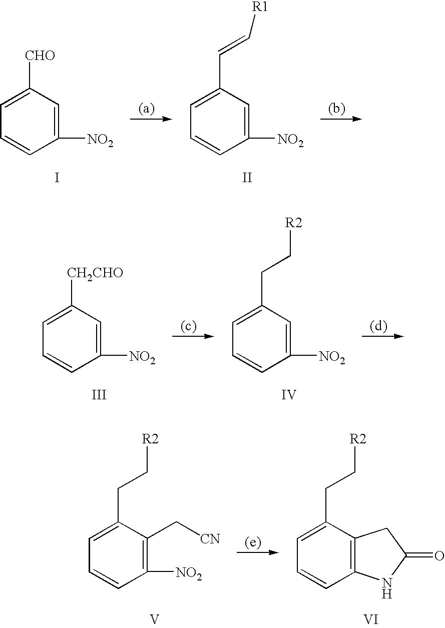

Wherein R1 is alkoxyl; R2 is amino, lower alkylamino, di-lower alkylamino, allylamino, diallylamino, N-lower alkyl-N-allylamino, benzylamino, dibenzylamino, phenethylamino, diphenethylamino, 4-hydroxyphenethylamino or di-(4-hydroxyphenethylamino).

### SUMMARY OF THE INVENTION

One object of the present invention is to provide a process for producing ropinirole with commercially available and inexpensive 3-nitrobenzaldehyde as the starting material.

Another object of the present invention is to provide a process for producing ropinirole that includes less reaction steps than heretofore known and that provides a higher yield than heretofore known.

These and other objects of the invention will become apparent by reference to the detailed description of the preferred embodiments of the invention.

### A General Description of Process

3-nitrobenzaldehyde is treated with Wittig reagent (methoxymethyl triphenyphosphonium chloride) followed by extraction with a hydrophilic organic solvent to give compound II. Compound II, is hydrolyzed under acidic condition and extracted with a hydrophilic organic solvent to produce the aldehyde (III), which in turn is subjected to reduction and amination followed by extraction with a hydrophilic organic solvent to yield the tertiary amine (IV). All these steps can be telescoped. Controlled nucleophilic substitution with 4-chlorophenoxyacetonitrile followed by extraction with a hydrophilic organic solvent gives only nitrile compound (V) at −10 to about −15° C. Finally the reduction and hydrolysis in the same step with palladium/C and sodium hydroxide followed by extraction with a hydrophilic organic solvent produced ropinirole in a yield of about 80%. The overall yield of this new 5-step synthetic route is about 36%.

### DETAILED DESCRIPTION OF PREFERRED EMBODIMENT OF THE INVENTION

The process of the present invention arrived at the desired compound of ropinirole without other isomers and by utilizing low reaction temperatures from ambient temperature to about −10 to −15 (° C.), an industrially amenable condition. Hence, a yield of 67% for isolated compound V is achieved.

Further, the invention process of preparing ropinirole utilizes the commercially available and cheap 3-nitrobenzaldehyde as the starting material-substantially less expensive than the isochroman and 2-methyl-3-nitrobenzoic acid as used in U.S. Pat. Nos. 4,452,808 and 5,336,781. (isochroman, Aldrich 25 g/$54.50; 3-nitrobenzaldehyde, Aldrich 500 g/$58.20).

Accordingly, the new synthetic route for producing ropinirole of the invention is brief and includes only 5 overall reaction steps. A higher product yield of about 36% is achieved. This represents a 50%-increase in yield comparing those achieved in the prior arts, 23% and 24% respectively.

Further still, for producing large quantities of ropinirole the first three reaction steps may be carried out in a single reaction vessel. The reaction scheme for preparing the ropinirole of the invention process is as follows:

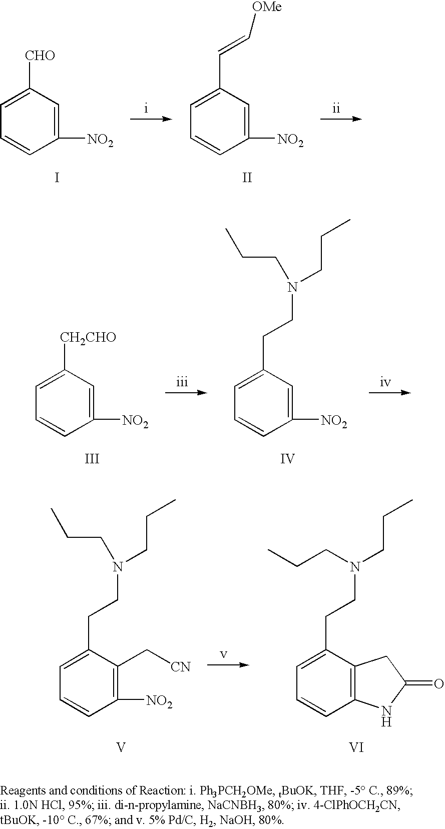

### EXAMPLES

### Preparation of β-methoxy-3-nitro-styrene (II)

To a suitable sized reaction vessel, methoxymethyltriphenyphosphonium chloride (4.75 g, 13.86 mmol) and anhydrous THF were added. The mixture was cooled down to 0° C. and potassium tert-butoxide (1.94 g, 17.33 mmol) was added to give a red suspension. The mixture was cooled down to −10° C. and a solution of 3-nitrobenzaldehyde (1.74 g, 11.55 mmol) in toluene was added during 30 minutes. The mixture was stirred for 1.0 hour, after which saturated ammonium chloride was added under 0° C., and the mixture was extracted twice with 20 mL of ethyl acetate. The combined organic layers were washed with brine and dried over anhydrous sodium sulfate, after which solvents were removed to give a yellow solid in 88% yield as a mixture of trans/cis isomers.

### Preparation of 2-(3-nitrophenyl)acetaldehyde (II)

To a solution of II (17.90 g, 0.10 mol) in THF was added 60 mL of 2N HCl and the mixture was heated at 80° C. THF was removed by vacuum and the residue was dissolved in ethyl acetate and water. The water layer was extracted with ethyl acetate. The combined organic layer was washed with 5% sodium bicarbonate and brine, and dried over anhydrous sodium sulfate. Thereafter, the solvents were removed to provide a product in 95% yield.

### Preparation of 2-(Di-n-propylamino)ethyl-nitrobenzene (IV)

To a solution of compound (III) (23.39 g, 0.14 mol) in THF-MeOH was added di-n-propylamine (24.70 mL, 0.18 mol) followed by the addition of sodium cyanoborate (12.47 g, 0.20 mol). The reaction mixture was stirred at ambient temperature. 50 mL of 1 N HCl was added to quench the reaction. The reaction was stirred and the crude product was purified to provide a product in 80% yield. ¹H-NMR (CDCl₃, 200 MHz): δ 8.10 (s 1H), 8.05 (d, 1H), 7.55 (d, 1H), 7.49 (t, 1H), 2.85 (dd, 2H), 2.70 (dd, 2H), 2.49 (t, 4H), 1.45 (m, 4H), 0.90 (t, 6H).

### Preparation of 2-(Di-n-propylamino)ethyl-6-nitro-phenylacetonitrile (V)

To a solution of compound IV (7.70 g, 31 mmol) and 4-chlorophenoxyacetonitrile (5.60 g, 33 mmol) in DMF was added potassium tert-butoxide (7.50 g, 93 mmol) under a temperature of −30° C. The mixture was stirred at that temperature until the reaction was completed. The pH was adjusted to between 1-2. The reaction mixture was extracted with EtOAc. The combined water layer was basified and extracted to give a crude product, which was chromatographed to provide pure V (8.70 g, 67%).

### Preparation of 2-(Di-n-propylamino)ethyl-2-oxoindole (VI, Ropirinole)

A mixture of compound V (0.51 g, 1.76 mmol) and 5% Pd/C (50 mg) was shaken under 14 psi of hydrogen at room temperature for 1 h. The reaction mixture was filtered and the filtrate was concentrated to give a residue. The residue was refluxed in a solution of 5 mL of 20% NaOH and 5 mL of dioxane over night. The mixture was cooled down and extracted three times with ethyl acetate (preferably 30 ml each). The organic layer was washed with water and diluted with 2-propanol, after which it was acidified to give a solid. The solid was collected to give the final product (VI) (500 mg, 80%). ¹H-NMR (d₆-DMSO, 200 MHz): δ 11.10 (bs, 1H), 10.50 (s, 1H), 7.20 (t, 1H), 6.80 (d, 1H), 6.70 (d, 1H), 3.60 (s, 2H), 3.20 (m, 2H), 3.10 (m, 6H), 1.80 (m, 4H), 0.90 (t, 6H); MS: 261 (M+1),

While ethyl acetate has been employed as the organic solvent, in the context of the invention, any hydrophilic organic solvent will suffice, as for example MTBE, dichloromethane, MeTHF and 2-butanone.

I claim:

1. A method for producing compounds of the formula: 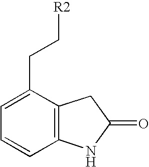
Wherein R2 is selected from the group consisting of amino, lower alkylamino, di-lower alkylamino, allylamino, diallylamino, N-lower alkyl-N-allylamino, benzylamino, dibenzylamino, phenethylamino, diphenethylamino, 4-hydroxyphenethylamino or di-(4-hydroxyphenethylamino);
comprising:
a) reacting 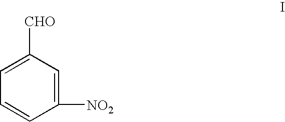
with Wittig Reagent extracting the formed mixture with a hydrophilic organic solvent and removing said solvent to produce 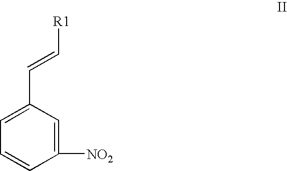
wherein R1 is alkoxyl;
b) hydrolyzing compound II under acidic conditions, dissolving the formed residue in a hydrophilic organic solvent and water, and removing said solvent to produce 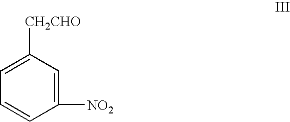
c) subjecting compound III to reduction and amination to produce the tertiary amine 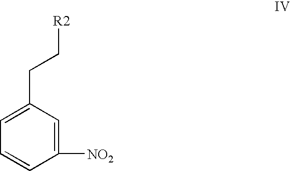
wherein R2 is as defined above;
d) reacting compound IV with
4-chlorophenoxyacetonitrile and extracting the formed reaction mixture with a hydrophilic organic solvent to create nucleophilic substitution to produce the nitrile compound 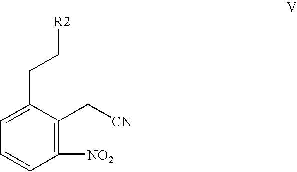
wherein R2 is as defined above; and
e) effecting reduction and hydrolysis in the same step with palladium/C and sodium hydroxide to produce 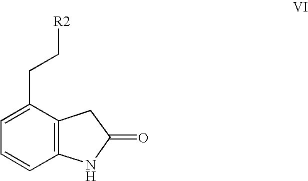
wherein R2 is as defined above.

2. The method of claim 1 wherein said hydrophilic organic solvent is selected from the group consisting of ethyl acetate, MTBE, dichloromethane, MeTHF and 2-butanone.

3. The method of claim 2 wherein step d) is conducted at a temperature of from about −10 to about −15° C.

4. The method of claim 3 wherein steps a) through c) are performed in a single reaction vessel.

5. A method for producing the compound Ropinirole of the formula: 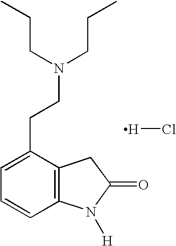
comprising:
a) reacting a mixture of methoxymethyltriphenyphosphonium chloride in anhydrous THF; adding a solution of 3-nitrobenzaldehyde in toluene, extracting the mixture with a hydrophilic organic solvent selected from the group consisting of ethyl acetate, MTBE, dichloromethane, MeTHF and 2-butanone, and removing said hydrophilic organic solvent to produce a mixture of trans/cis isomers of: 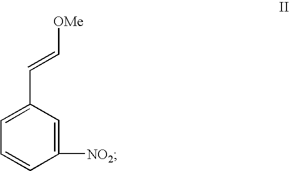
b) adding HCL to a mixture of compound II in THF, heating said mixture, removing THF and dissolving the residue in said hydrophilic organic solvent and water mixture, removing said hydrophilic organic solvent and water mixture to obtain: 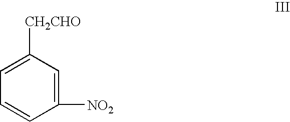
c) adding di-n-propylamine to a solution of compound III in THF-MeOH followed by addition of sodium cyanoborate to produce: 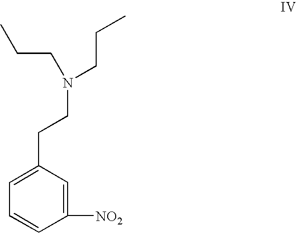
d) adding potassium tert-butoxide under −30° C. to a solution of compound IV and 4-chlorophenoxyacetonitrile in DMF, stirring to complete reaction, adjusting pH to between about 1 and 2, extracting the reaction mixture with said hydrophilic organic solvent, and alkalizing the mixture to produce: 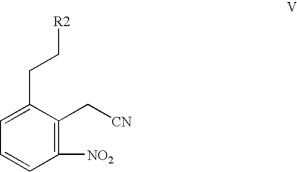
and
e) shaking a mixture of compound V and Pd/C in hydrogen at room temperature, filtering said mixture and forming a residue from the filtrate, refluxing said residue in a solution of NaOH and dioxane, washing an organic layer of said residue with water and 2-propanol, and acidifying to produce the solid compound:

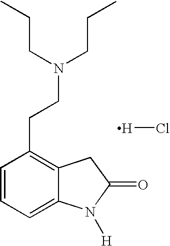

6. The method of claim 5 wherein said hydrophilic organic solvent is ethyl acetate.

7. The method of claim 5 wherein said hydrophilic organic solvent is MTBE.

8. The method of claim 5 wherein said hydrophilic organic solvent is dichloromethane.

9. The method of claim 5 wherein said hydrophilic organic solvent is MeTHF.

10. The method of claim 5 wherein said hydrophilic organic solvent is 2-butanone.

11. The method of claim 6 wherein in step e) shaking said mixture of compound IV and Pd/C is in hydrogen at about 14 psi.
